(19) United States
(12) Patent Application Publication
(10) Pub. No.: US 2020/0253598 A1
(43) Pub. Date: Aug. 13, 2020

(54) Suture Anchor Implantation System

(71) Applicant: S. Wendell Holmes, JR., Lexington, SC (US)

(72) Inventor: S. Wendell Holmes, JR., Lexington, SC (US)

(21) Appl. No.: 16/787,923

(22) Filed: Feb. 11, 2020

Related U.S. Application Data
(60) Provisional application No. 62/804,578, filed on Feb. 12, 2019.
(60) Provisional application No. 62/826,472, filed on Mar. 29, 2019.

(51) Int. Cl. A61B 17/04

(52) U.S. Cl. CPC A61B 17/0401; A61B 2017/0496; A61B 17/0469; A61B 2017/0409

(57) ABSTRACT

A system for securing a suture in boney tissue with an anchor. A handle member defines a central bore generally configured to receive a first suture. A driver is connected to the handle and defines a central bore in communication and co-axial with the central bore of the handle which is configured to receive the first. The handle is configured to rotate the driver, and the driver, in turn, rotate and seat the anchor. A retainer proximate the handle holds the first suture, upon a loop of the first suture grasping the second suture prior to the second suture being secured using the retainer. A collar adjacent the handle defines a central bore coaxial with the central bore of the handle configured to receive the driver, and another retainer is provided on the collar for holding the second suture.

### FIELD

The present disclosure relates generally to methods and configurations for anchoring a suture to bone. More specifically, certain aspects of the disclosure relate to apparatuses, methods and systems for anchoring a suture within a hole in boney tissue, wherein no suture knot is required and wherein only an anchor member and the anchored suture remain in the hole.

In arthroscopic orthopaedic surgery, it is often necessary to reattach soft tissue to a boney structure. This occurs frequently in the shoulder and other tissues. In these situations where soft tissue is being reattached to bone, it is useful to use a threaded suture anchor device to make the necessary repair. A right-hand helically threaded suture anchor is typically placed into the bone and will not ordinarily pull out. Such suture anchor typically has sutures of its own that can be used to effectively reattach and tie down soft tissue to the bone. This reattachment allows the soft tissue to eventually heal to the bone, and during the process of healing, the suture anchor holds the tissue stationary on the bone.

A suture anchor preferably resists gapping at the tissue bone interface and also resists cyclic loosening of the suture and the tissue from the bone. Anchors can be made of metal, plastic, or absorbable plastics, and designs vary with respect to the number of sutures provided and with respect to whether or not such sutures are allowed to slide within the anchor.

In the normal use of suture anchors, the sutures are passed through the tissue and then knots are tied which allow the anchor to be held into place. While knotless anchors have been produced, a disadvantage can be that certain knotless suture anchors may require items such as eyelets, grommets, spacers, etc. to be left behind in the boney tissue after implantation of the suture anchor. Additionally, certain devices for implanting an anchor include an aperture for retaining the suture thread or tape (both collectively referred to herein as “suture” or “sutures) to be seated in the bone. Such apertures can themselves pose concern if not sized sufficiently to carry the suture to be anchored and/or if too large to pass freely in the hole in the bone used to seat the anchor.

Accordingly, suture anchor implantation systems and methods which eliminate the need to tie knots and which do not require items such as eyelets, guides, spacers, etc. to be left behind in the boney tissue after implantation of the suture anchor would be desirable.

### SUMMARY

It would be desirable to provide an apparatus and method that address at least some of the issues discussed above, as well as other potential issues. Moreover, it would be beneficial to furnish system for anchoring a suture within a hole in boney tissue, wherein no suture knot is required.

It is noted here that Applicant's pending U.S. Patent Application Nos. 61/605,962, filed Mar. 2, 2013; Ser. No. 14/382,234, filed Aug. 29, 2014 (now, U.S. Pat. No. 10,542,966, issued Jan. 28, 2020); 62/804,578, filed Feb. 12, 2019; 62/826,472, filed Mar. 29, 2019; and Ser. No. 16/723,200, filed Dec. 20, 2019 are hereby incorporated herein in their entirety by reference.

Accordingly, apparatuses and methods are disclosed for anchoring a suture within a hole in boney tissue substantially as shown in and/or described in connection with at least one of the figures, and as set forth more completely in the claims.

More specifically, examples of the present disclosure are generally directed to apparatuses, systems, and methods for anchoring a suture within a hole in boney tissue, wherein no suture knot is required and wherein only a threaded anchor member and the anchored suture remain in the hole.

In one exemplary implementation disclosed herein, a system is provided for receiving a plurality of sutures and for securing at least one thereof in boney tissue using an anchor, the system including an elongated handle member defining a central bore extending therethrough generally centered about a central axis. The handle member has a first end and a second end generally opposite the first end, and the central bore is configured to receive a first suture of the plurality of sutures. An elongated driver portion is connected to the handle and defines a central bore extending therethrough generally centered about the central axis and in communication with the central bore of the handle, the central bore of the driver portion being configured to receive the first suture. At least one first cleat is proximate the first end of the handle and is configured to receive and hold the first suture, and the handle is configured to rotate the driver portion upon rotation of the handle. An elongated collar configured to receive the driver portion is proximate the second end of the handle and defines a central bore extending therethrough generally centered about the central axis. And, at least one second cleat is provided on the collar which is configured to receive and hold a second suture of the plurality of sutures.

In some implementations, the driver includes a longitudinally-extending profile configured for securely engaging the anchor for rotation with the driver, the profile including, in some implementations, a cross-section including at least one of a group consisting of star shape, a decagon shape, nonagon shape, an octagon shape, heptagon shape, hexagon shape, square shape, triangle shape, oval shape, symmetrical shape, asymmetrical shape, and polygonal shape.

In some implementations, the collar is configured to selectively move axially with respect to the driver and/or other implementations may include a circumferentially extending ring may be provided on the driver and a releasable retainer provided to selectively engage the ring, wherein the collar is restrained from moving axially with respect to the driver but free to remain stationary while the driver rotates

In some implementations, the driver has an extreme end opposite the second end of the handle and the first suture has a first leg and a second leg extending from the central bore of the handle and proximate the first cleat, and an intermediate portion of the first suture is formed in a loop extending from the extreme end of the driver. In other implementations, the driver has an extreme end opposite the second end of the handle and a generally cylindrical leading portion adjacent the extreme end.

Further implementations include a sleeve having an opening in communication with the central bore of the handle, and the first cleat is connected to the sleeve, and wherein the sleeve is configured to be free to remain stationary while the handle and the driver rotate.

Another exemplary implementation of the present disclosure involves a system for receiving a plurality of sutures and for securing at least one thereof in boney tissue using an anchor, wherein the system includes an elongated handle having first end and a second end generally opposite the first end and defining a central bore extending therethrough generally centered about a central axis. The central bore is configured to receive a first suture of the plurality of sutures. An elongated driver is connected to the handle and defines a central bore extending therethrough generally centered about the central axis and in communication with the central bore of the handle, the central bore of the driver being configured to receive the first suture, and the handle being configured to rotate the driver upon rotation of the handle. An elongated collar proximate the second end of the handle is configured to receive the driver and defines a central bore extending therethrough generally centered about the central axis.

Other exemplary implementations include providing at least one first releasable fastener proximate the first end of the handle configured to receive and hold the first suture, at least one resilient band provided on the collar configured to receive and hold a second suture of the plurality of sutures, a loop interposed between the resilient band and the collar configured to receive and hold a second suture of the plurality of sutures, the elongated driver having a free end with a generally circular end face defining a textured surface profile (such as a grooves, cross-hatching, roughening, etc.) configured to selectively engage the second suture and/or the elongated driver having a biased fastener (which could be a ball detent) configured to selectively engage and retain an anchor on the elongated driver.

In another exemplary implementation, a sleeve is provided having an opening in communication with the central bore of the handle and also a retainer (which could be an O-ring) configured to retain the sleeve on the driver while allowing the sleeve to rotate and to move axially with respect to the driver, wherein the sleeve is configured to be free to remain stationary while the handle and the driver rotate.

Other exemplary implementations of the present disclosure include methods for implanting a threaded suture anchor for accommodating a plurality of sutures and for securing the anchor in boney tissue using the anchor and a tendon to the anchor. Such the methods include: (a) providing an elongated driver connected to a handle, each having a central bore extending therethrough in communication with one another and configured to receive a first suture of the plurality of sutures; (b) forming a loop in the first suture extending outwardly from a free end of the driver and passing the two legs of the first suture through the central bores of the driver and handle; (c) passing a second suture of the plurality of sutures through the tendon with a suture passer to result in two legs of the second suture; (d) passing the two legs of the second suture through the loop; (e) applying tension on the loop by pulling from adjacent the end of the handle opposite the free end of the driver on at least one of the two legs of the first suture; (f) attaching the anchor to the driver; (g) inserting the free end of the driver, the loop, the two legs of the second suture, and the anchor into a hole in the bone; and (h) simultaneously applying tension on the loop and rotating the handle to screw the anchor into the hole and to secure the two legs of the second suture to the bone.

Other exemplary implementations of methods include maintaining tension in one or more of the legs of the first suture using a least one first releasable fastener and/or cleat proximate the first end of the handle and/or retaining one or more of the legs of the second suture to the handle and/or collar (which could include use of a cleat and/or a resilient band).

Another exemplary implementation of methods include providing a loop interposed between a resilient band on the collar adjacent the handle configured to receive and hold one or more legs of the second suture and to facilitate pulling such leg(s) beneath the resilient band to permit the resilient band to secure or grip the leg(s) against the handle/collar.

The features, functions and advantages discussed herein may be achieved independently in various example embodiments or may be combined in yet other example embodiments further details of which may be seen with reference to the following description and drawings.

### BRIEF DESCRIPTION OF THE DRAWINGS

The drawings referenced herein form a part of the specification. Features shown in the drawings are meant as illustrative of some, but not all, embodiments of the present disclosure, unless otherwise explicitly indicated, and implications to the contrary are otherwise not to be made. Although in the drawings like reference numerals correspond to similar, though not necessarily identical, components and/or features, for the sake of brevity, reference numerals or features having a previously described function may not necessarily be described in connection with other drawings in which such components and/or features appear.

FIG. 1 is an elevational view of an exemplary implementation of a suture anchor implantation system in accordance with the present disclosure;

FIG. 2 is a perspective view of an exemplary implementation of a suture anchor implantation system in accordance with the present disclosure;

FIG. 3 is a partial perspective view of an exemplary implementation of a suture anchor implantation system in accordance with the present disclosure;

FIG. 4 is a perspective view of an alternative end face of a driver of an exemplary implementation of a suture anchor implantation system in accordance with the present disclosure;

FIG. 5 is a perspective view of an exemplary implementation of a suture anchor implantation system in accordance with the present disclosure, wherein a handle and attached driver carries a suture anchor for relative axial movement and in central bores thereof a first suture secured to a cleat of a sleeve on the upper end of the handle, boney tissue with a predrilled and/or tapped hole therein, soft tissue with two legs of a second suture extending therefrom and captured intermediately by a loop of the first suture extending from the lower, extreme end of the driver and cleated in a second cleat of a collar, which partially covers the driver and which can selectively move axially with respect to the driver to advance the anchor during implantation thereof;

FIG. 6A is an elevational view of another exemplary implementation of a suture anchor implantation system in accordance with the present disclosure;

FIG. 6B is a partial is a partial perspective view of the free end of an exemplary implementation of a suture anchor implantation system in accordance with the present disclosure;

FIG. 7 is a perspective view of another exemplary implementation of a suture anchor implantation system in accordance with the present disclosure;

FIG. 8A is a partial is a partial perspective view of an exemplary implementation of a suture anchor in accordance with the present disclosure;

FIG. 8B is a partial is a partial perspective view of another implementation of a suture anchor in accordance with the present disclosure;

FIG. 9 is a perspective view of a further exemplary implementation of a suture anchor implantation system in accordance with the present disclosure; and

FIG. 10 is a perspective view of still further exemplary implementation of a suture anchor implantation system in accordance with the present disclosure.

### DETAILED DESCRIPTION

Some examples of the present disclosure will now be described more fully hereinafter with reference to the accompanying drawings, in which some, but not all examples of the disclosure are shown. Indeed, various aspects of the disclosure may be embodied in many different forms and should not be construed as limited to the examples set forth herein. Rather, these examples are provided so that this disclosure will be thorough and complete and will fully convey the scope of the disclosure to those skilled in the art. Like reference numerals refer to like elements throughout.

As used herein, “and/or” means any one or more of the items in the list joined by “and/or”. As an example, “x and/or y” means any element of the three-element set, e.g., {(x), (y), (x, y)}. Additionally, as used herein, the terms “example” and “exemplary” means serving as a non-limiting example, instance, or illustration. Moreover, as used herein, the term, for example, or “e.g.,” introduces a list of one or more non-limiting examples, instances, or illustrations.

Referring more particularly to the drawings, examples of the present disclosure include apparatus, methods and systems for anchoring a suture within a hole in boney tissue.

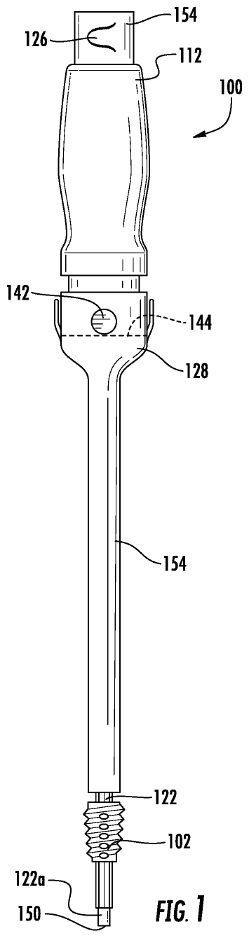

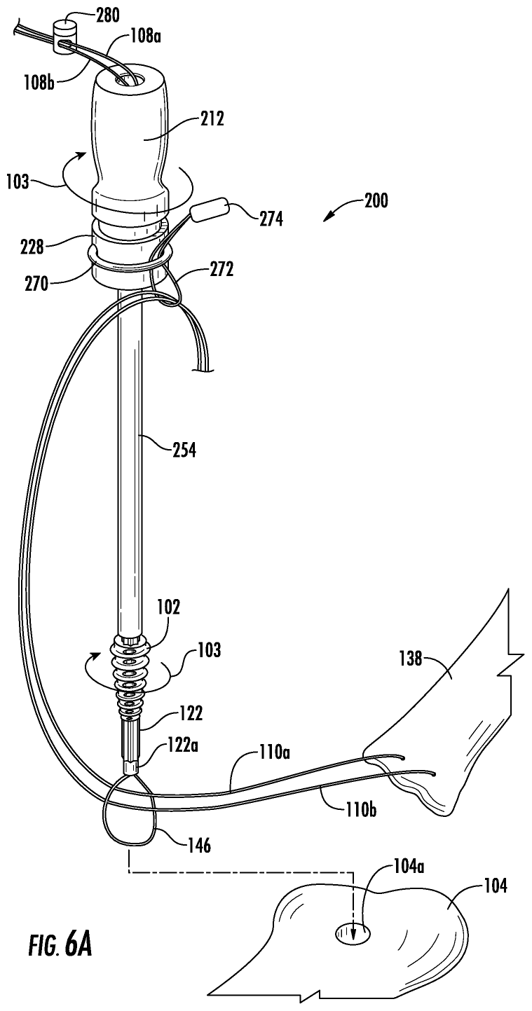

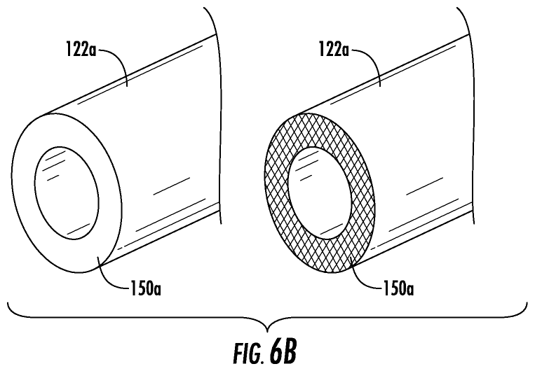

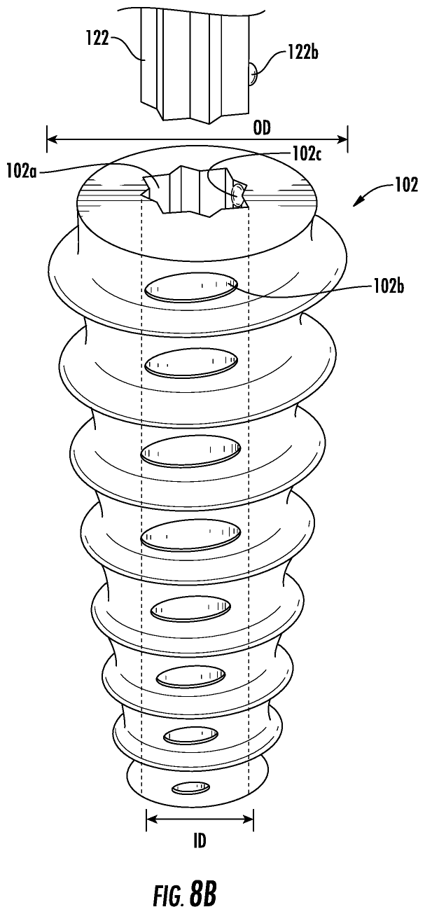

Turning to FIGS. 1-4, one exemplary implementation of a suture anchor implantation system, generally 100, disclosed herein. System 100 accommodates a plurality of sutures, and in one implementation, first suture 108 and second suture 110, and for securing at least one of sutures 108, 110 in boney tissue 104 using a suture anchor 102. Anchors 102 are typically made of various materials including PEEK, metal, carbon fiber, and absorbable materials (polymers including a bio-composite polymer). The pores or slots 102b in the anchors encourage biologic growth. A typical length of an anchor is 5-25 mm. Inner cross section diameters ID (FIGS. 8A and 8B) can be of various designs including star drive, hexagonal, etc. Outer diameters OD (FIGS. 8A and 8B) can vary widely depending on application—typically 1 mm to 15 mm. Most common diameters would be 2.0 to 5.0 mm.

System 100 includes an elongated handle member, generally 112, defining a central bore 114 extending therethrough generally centered about a central axis extending through the handle 112, driver portion, generally 122, and collar, generally 128, of system 100. The handle member 112 has a first end 118 and a second end 120 generally opposite the first end 118, and the central bore 114 is configured to receive first suture 108.

The elongated driver portion 122 is connected to the handle 112 and defines a central bore 114 extending therethrough generally centered about the central axis and in communication with the central bore 114 of the handle 112, the central bore 124 of the driver portion 122 being configured to receive the first suture 108. At least one first cleat 126 is proximate the first end 118 of the handle 112 and is configured to receive and hold the first suture 108. The handle 112 is configured to rotate the driver portion 122 upon rotation of the handle 112.

The elongated collar 128 is configured to receive the driver portion 122, is proximate the second end 120 of the handle 112, and defines a central bore 132 extending therethrough generally centered about the central axis. At least one second cleat 134 is provided on the collar 128, which is configured to receive and hold a second suture 110 of the plurality of sutures.

In some implementations, the driver 122 includes a longitudinally-extending profile configured for securely engaging the anchor for rotation with the driver, the profile including, in some implementations, a cross-section including at least one of a group consisting of star shape 140, and/or, although not shown, a decagon shape, nonagon shape, an octagon shape, heptagon shape, hexagon shape, square shape, triangle shape, oval shape, symmetrical shape, asymmetrical shape, polygonal shape or any other suitable shape.

In some implementations, the collar 128 is configured to selectively move axially with respect to the driver 122 and/or other implementations may include a circumferentially extending ring 142 (FIG. 1) may be provided on the driver and a releasable retainer 144 provided to selectively engage the ring 142, wherein the collar 128 is selectively restrained from moving axially with respect to the driver but free to remain stationary while the driver rotates.

In some implementations, the driver 122 has an extreme, or free, end 150 opposite the second end 120 of the handle 112 and the first suture 108 has a first leg 108a and a second leg 108b extending from the central bore of the handle and proximate the first cleat 126, and an intermediate portion of the first suture is formed in a loop 146 extending from the extreme end of the driver.

In other implementations, the driver 122 has an extreme end 150 opposite the second end of the handle and a generally cylindrical-exterior leading portion 122a adjacent the extreme end.

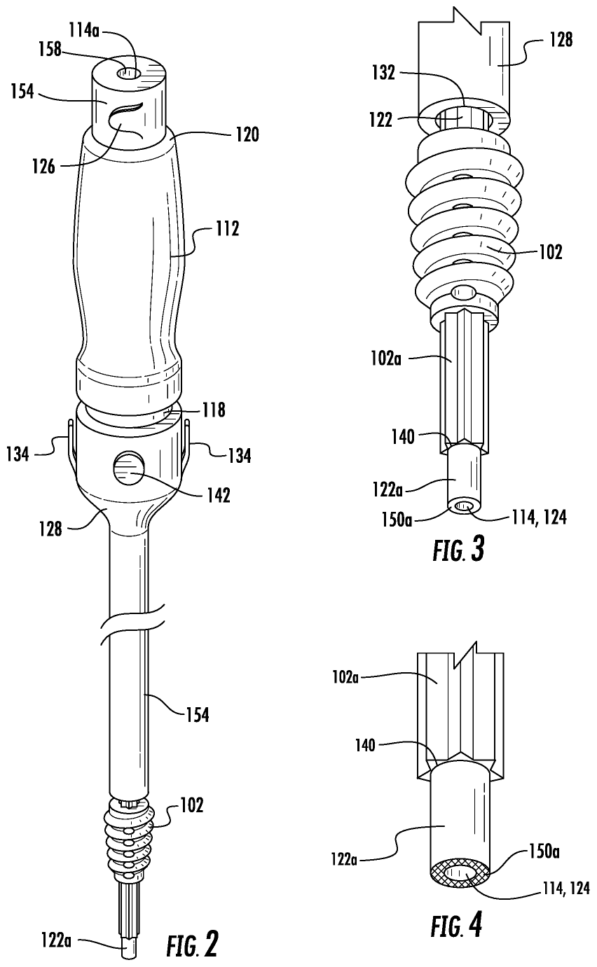

Further implementations include a sleeve 154 having an opening 158 (FIG. 2) in communication with the central bore of the handle 112, and the first cleat 126 is connected to the sleeve 154, and wherein the sleeve 154 is configured to be free to remain stationary while the handle 112 and the driver 122 rotate.

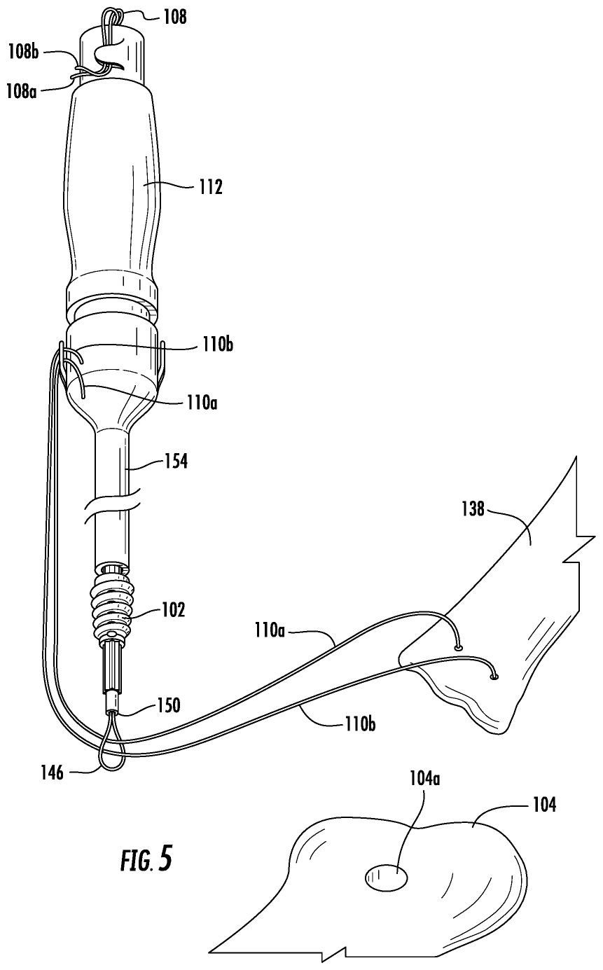

Implementations of the present disclosure include methods, processes, and techniques for implanting a suture anchor, including passing second suture 110 through a tendon 138, which could be a rotator cuff tendon, or some other tendon or soft tissue. This can be done with the second suture having a needle attached to one leg 110a of suture 110, which is used to draw suture 110 through the tendon or other soft tissue from a first side of the tissue to a second side of the tissue, and then from the second side of the tissue back through such tissue to the first side, thereby leaving first and second legs 110a, 100b of the suture 110 extending from the first side of the such tendon or soft tissue. The needle can then be removed from suture 110 and perhaps discarded. The process also includes drilling and/or tapping a hole 104a in boney tissue, or bone, 104. The suture may also be passed via multiple types of suture passers designed for arthroscopic or open surgery. As shown in FIGS. 3, 4, and 6B, end 150 defines a circular end face 150a that can be smooth, or alternately can include a profiled surface, such as a grooved, cross-hatched, textured, roughened, etc. surface to facilitate gaining purchase on sutures 110 so as to reduce the likelihood of sutures 110 migrating away from face 150a when installing the anchor in the hole 104a.

First suture 108 is disposed within central bores 114 and 124 of handle 112 and driver portion 122 in a manner that two legs 108a, 108b of suture 108 extend through an upper opening of central bore 114 of handle 112, and an intermediate portion of suture 108 forms a loop 146, which extends outwardly from the extreme tip 150 of driver 122. Both legs of second suture 110 are passed through the suture loop 146, and tension is applied to suture loop 146 by pulling on legs 108a and 108b of first suture 108 through central bore 114 at the upper end 120 of handle 112, with suture 108 passing through central bore 114.

For example, a surgeon's left hand could hold handle 112, while his or her right hand pulls the suture loop 146 tight around the two legs of suture 110 by pulling legs 108a and 108b, such that the suture loop 146 hold suture tape 110 securely. The leading end of the anchor 102 can then be inserted into the hole 104a within bone 104, and the suture 110 grasped and cinched up against the extreme end 122a of driver 112 via loop 146, by maintaining tension within suture legs 108a, 108b. Tension is also maintained in suture 110 by pulling handle 112 away from the soft tissue where the suture 110 is affixed. Such tension, once obtained, that allows for the free ends of the two legs of suture tape to be cleated on second cleat 134.

The anchor includes an interior profile 102a (FIG. 3) which corresponds to the longitudinally extending exterior profile of driver 112. For example, as shown in the figures, the exterior profile of driver 112 could be a star shape cross-section or a Torx cross-section, or some other suitable similar profile, and the interior portion of anchor 102 has a mating profile. This allows anchor 102 to move axially on driver 112. Thus, as driver 122 is rotated by handle 112, anchor 102 is rotated in unison (in the direction shown by arrow 103 if a right-hand threaded anchor is used (although left-hand threaded anchors could also be used if desired)), such that upon insertion of anchor 102 into the hole 104a, and rotation of handle 112, anchor 102 is turned, i.e., screwed, into the hole 104a. Because the leading end 150 of the driver 122 is inserted into the hole, and consequently, because suture 110 is held within loop 146 formed by suture 108, suture 110 is also carried into the hole and is positioned at or near the bottom of the hole, upon driver 122 being advanced all the way into the hole. Note that driver 122 includes the generally smooth, cylindrical leading, or pilot, section 122a to facilitate smooth entry into the hole, particularly if the hole is tapped. In one implementation, the driver could be approximately 40 millimeters in length, and anchor 102 approximately 20 millimeters in length, although many other variations in the size and length of anchor and pilot section 122a could also be used.

In another exemplary implementation, a tap-in anchor could be used to secure sutures 110 within hole 104a, with proper tension being maintained in the sutures 108, 110, with system 100 while handle 112 is driven with a mallet to force the anchor into the hole 104a. In such implementation, the threads on the anchor do not need to be helically configured but instead could be of stacked, parallel rib design (not shown).

Once the pilot portion 122a of driver 112 is seated within the base of hole 104a, collar 128, because it is configured to move axially along driver shaft 112, allows for the surgeon to simultaneously maintain the desired tension within sutures 110 while pressing anchor 102 into the hole, via advancing collar 128 forward and against anchor 102. Continued rotation of driver 122 causes anchor 102 to advance further downwardly to threadingly advance further downward within the hole until it ultimately seats in a manner sufficient to securely retain sutures 110 within the hole. The collar 128 can also be restrained from moving axially by actuating releasable retainer 142 (FIG. 2), if desired, which through interaction with ring 144, allows collar 128 to remain stationary if held by the surgeon as handle 112 is rotated.

Upon adequately tension being obtained in suture 110, by the surgeon pulling suture 110 taught and/or to some other desired tension, anchor 102 is screwed into the tape. Note that once desired tension is achieved in suture 108, it can be secured by inserting it within cleat 134 on collar 128.

After the anchor 102 has been screwed in the bone, that portion of sutures 108 extended between anchor 102 and cleat 134 can be cut. Then, suture 108 can be removed from cleat 126 and one leg of suture 108 pulled such that the suture loop 146 is removed from its entirety from both the hole and from system 100, i.e., from handle 112 and driver 122.

From the foregoing, it can be seen that system 100 allows a suture to be secured in a bone without using knots, thereby eliminating knots which can irritate the patient, slip, etc., and also eliminating the time it takes and clearance required to tie such knots.

Additionally, while certain other anchor implantation techniques may leave behind eyelet stays in the hole 104a of the bone, system 100, leaves only the anchor suture 110 behind in the hole, since the suture loop 146 is also removed from the hole through the central bores of driver 122 and handle 112.

Further, eyelets used in other devices are typically of a fixed size, thereby providing an inherent limit as to how many sutures and/or the size of sutures which can pass through such eyelet, or aperture. Because of the variability of size of suture loop 146, many different variations in sizes and numbers of sutures and/or suture tapes can be accommodated by suture loop 146.

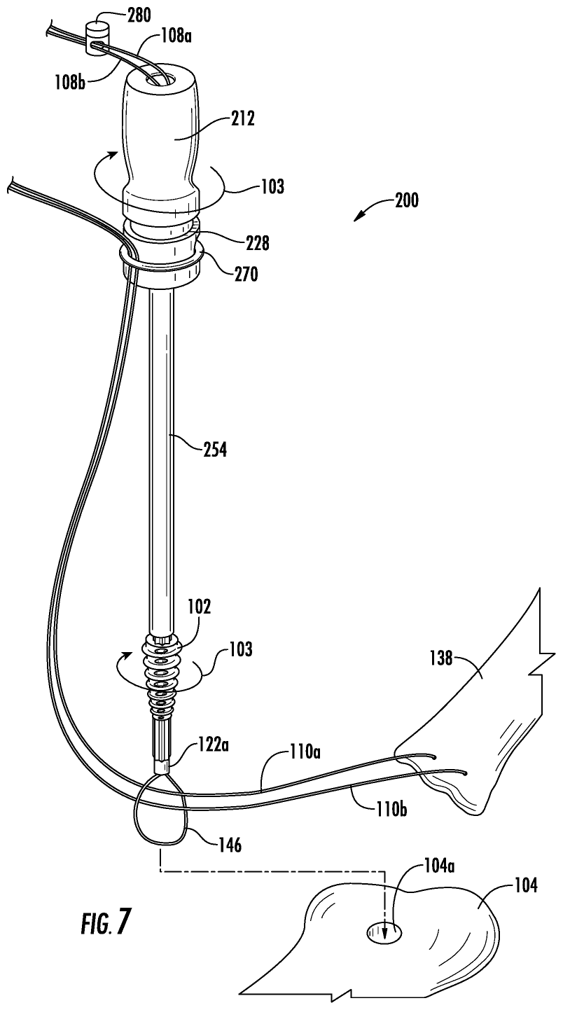

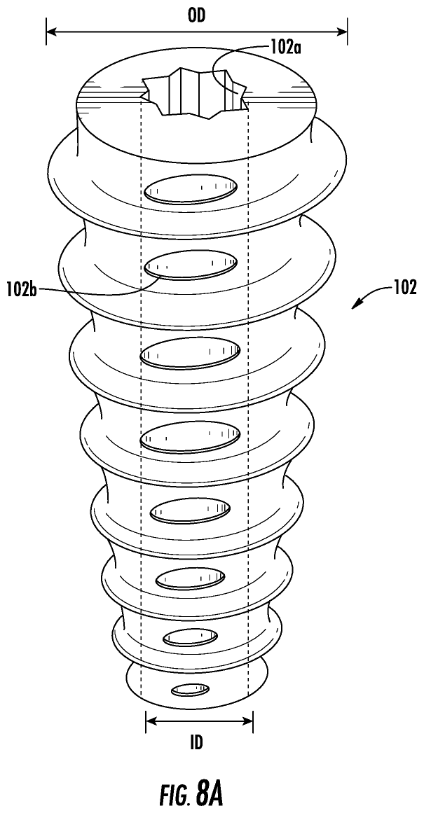

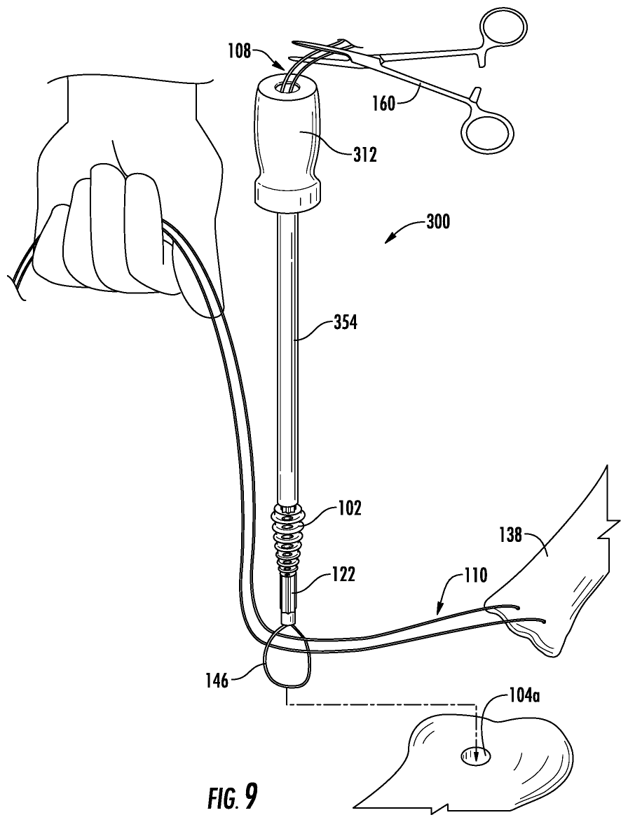

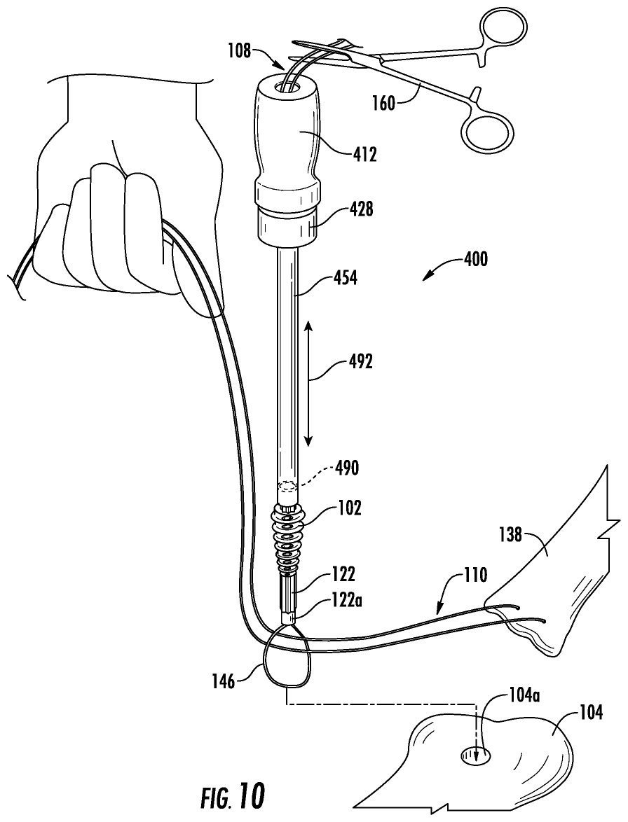

Turning to FIGS. 6A-10, other exemplary implementations of suture anchor implantation systems disclosed herein are shown, including system 200, shown in FIGS. 6A, 6B, and 7; system 300, shown in FIGS. 9 and 6B; and system 400, shown in FIGS. 10 and 6B. In those figures, like reference numerals correspond to similar, though not necessarily identical, components and/or features already described herein, and for the sake of brevity, such reference numerals or features having a previously described function may not necessarily be described in connection with other drawings in which components and/or features appear regarding systems 200, 300, and 400.

System 200, while having similarities with the system 100, embodies several aspects which could be used instead of or in addition to aspects already disclosed above regarding system 100. For example, system 200 includes a retaining band 270, which could be used in addition to or instead of a cleat, such as cleat 134, such as an elastic strap, band, or O-ring ring, carried on collar 228. Retaining band 270 is used to retain tension with in sutures 110 during anchoring of sutures 110 within boney tissue 104. As shown in FIG. 7, wherein sutures 110 are shown as having been inserted beneath retaining band 270 and are securely retained against collar 228. Later, when tension is to be applied to sutures 110, the sutures 110 are gripped sufficiently by retaining band 270 to reduce or prevent slippage or relative movement between sutures 110 and collar 228 and band 270.

To facilitate insertion of sutures 110 beneath band 270, a loop 272 can be preloaded beneath band 270, in a manner shown in FIG. 6A. A tab 274 connected to loop 272 allows for sutures 110 to be loaded or inserted between band 270 and collar 228 by pulling tab 274 upwardly, which in turn, pulls loop 272 and sutures 110 upwardly through the interface between band 270 and a collar 228.

System 200 also includes use of a retainer device 280, which could be used in addition to or instead of a cleat, such as cleat 126. In an embodiment shown in FIGS. 6A and 7, the retainer 280 is a spring loaded barrel clip having a passage through which sutures 108 pass. To tension sutures 110 within loop 146 of sutures 108, sutures 108 would be pulled through the opening 114 of the handle 212, and once proper tension was achieved, fastener 280 is advanced to be in engagement with the end face of handle 212 in order to maintain tension and sutures 108.

As shown in FIG. 8, systems 100, 200, 300, and 400 could include a driver 122 having a retaining device to securely retain an anchor 102 in a selectively releasable manner. For example, and without limitation, a ball detent 122b could be used for being received in a semi-hemispherical indention 102c within anchor 102. This retaining device facilitates retaining anchor 102 on driver 122 and preventing it from accidentally becoming detached or otherwise separated from driver 122.

FIG. 9 illustrates system 300, which does not include retaining band 270, retainer 280, or cleats. In this exemplary implementation, tension is maintained within sutures 108 with standard forceps 160, and the surgeon's own hand is used to maintain desired tension in suture 110 during attachment of sutures 110 to bone 104.

System 400 is similar to system 300, in that it does not require cleats, retaining band 270, or retainer 80, but instead relies on tension being maintained in sutures 108,110 with a clamping device such as forceps 160 and the surgeon's hand, respectively. However, system 400 includes a collar 428 attached to a driver sleeve 454 encircling driver 122 which is allowed to move axially upwardly and downwardly with respect to driver 122. Driver sleeve 454 includes in an interior portion thereof a retainer such as an O-ring 490, which, while permitting axial movement of sleeve 454 and rotation of sleeve 454 with respect to driver 122, otherwise prevents sleeve 454 from becoming separated or detached from system 400. Note that the ability of sleeve 454 to move axially allows the surgeon to force sleeve 454 downwardly against the top of anchor 102 during installation of anchor 102 within hole 104a of honey tissue 104.

Many modifications and other examples of the disclosure set forth herein will come to mind to those skilled in the art to which this disclosure pertains, having the benefit of the teachings presented in the foregoing descriptions and the associated drawings. Therefore, it is to be understood that the disclosure is not to be limited to the specific examples disclosed and that modifications and other embodiments are intended to be included within the scope of the appended claims.

Moreover, although the foregoing descriptions and the associated drawings describe aspects of the disclosure in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

What is claimed is:

1. A system is provided for receiving a plurality of sutures and for securing at least one thereof in boney tissue using an anchor, the system comprising:
an elongated handle having first end and a second end generally opposite the first end and defining a central bore extending therethrough generally centered about a central axis, the central bore being configured to receive a first suture of the plurality of sutures;
an elongated driver connected to the handle and defining a central bore extending therethrough generally centered about the central axis and in communication with the central bore of the handle, the central bore of the driver being configured to receive the first suture;
at least one first releasable fastener proximate the first end of the handle configured to receive and hold the first suture, and the handle being configured to rotate the driver upon rotation of the handle;
an elongated collar proximate the second end of the handle configured to receive the driver and defining a central bore extending therethrough generally centered about the central axis; and
at least one resilient band provided on the collar configured to receive and hold a second suture of the plurality of sutures.

2. The system of claim 1, further comprising the driver having a longitudinally-extending profile configured for securely engaging the anchor for rotation with the driver.

3. The system of claim 2, further comprising the driver having a longitudinally-extending profile of a cross-section including at least one of a group consisting of star shape, a decagon shape, nonagon shape, an octagon shape, heptagon shape, hexagon shape, square shape, triangle shape, oval shape, symmetrical shape, asymmetrical shape, and polygonal shape.

4. The system of claim 1, further comprising the collar being configured to move axially with respect to the driver.

5. The system of claim 1, further comprising a loop interposed between the resilient band and the collar configured to receive and hold a second suture of the plurality of sutures.

6. The system of claim 1, further comprising the elongated driver having a biased fastener configured to selectively engage and retain an anchor on the elongated driver.

7. A system is provided for receiving a plurality of sutures and for securing at least one thereof in boney tissue using an anchor, the system comprising:
an elongated handle having first end and a second end generally opposite the first end and defining a central bore extending therethrough generally centered about a central axis, the central bore being configured to receive a first suture of the plurality of sutures;
an elongated driver connected to the handle and defining a central bore extending therethrough generally centered about the central axis and in communication with the central bore of the handle, the central bore of the driver being configured to receive the first suture, and the handle being configured to rotate the driver upon rotation of the handle; and
an elongated collar proximate the second end of the handle configured to receive the driver and defining a central bore extending therethrough generally centered about the central axis.

8. The system of claim 7, further comprising at least one first releasable fastener proximate the first end of the handle configured to receive and hold the first suture.

9. The system of claim 8, further comprising at least one resilient band provided on the collar configured to receive and hold a second suture of the plurality of sutures.

10. The system of claim 7, further comprising the collar being configured to move axially with respect to the driver.

11. The system of claim 7, further comprising a loop interposed between the resilient band and the collar configured to receive and hold a second suture of the plurality of sutures.

12. The system of claim 7, further comprising the elongated driver having a free end with a generally circular end face defining a textured surface configured to selectively engage the second suture.

13. The system of claim 7, further comprising the elongated driver having a biased fastener configured to selectively engage and retain an anchor on the elongated driver.

14. The system of claim 7, further comprising:
the collar being configured to selectively move axially with respect to the driver;
a circumferentially extending ring on the driver; and
a releasable retainer configured to selectively engage the ring, wherein the collar is restrained from moving axially with respect to the driver but free to remain stationary while the driver rotates.

15. The system of claim 7, further comprising:
the driver having an extreme end opposite the second end of the handle; and
a generally cylindrical leading portion adjacent the extreme end.

16. The system of claim 7, further comprising:
a sleeve having an opening in communication with the central bore of the handle;
a retainer configured to retain the sleeve on the driver while allowing the sleeve to rotate and to move axially with respect to the driver; and
wherein the sleeve is configured to be free to remain stationary while the handle and the driver rotate.

17. A method for implanting a threaded suture anchor for accommodating a plurality of sutures and for securing the anchor in boney tissue using the anchor and a tendon to the anchor, the method comprising:
providing an elongated driver connected to a handle, each defining a central bore extending therethrough in communication with one another and configured to receive a first suture of the plurality of sutures;
forming a loop in the first suture extending outwardly from a free end of the driver and passing the two legs of the first suture through the central bores of the driver and handle;
passing a second suture of the plurality of sutures through the tendon with a suture passer to result in two legs of the second suture;
passing the two legs of the second suture through the loop;
applying tension on the loop by pulling from adjacent the end of the handle opposite the free end of the driver on at least one of the two legs of the first suture;
attaching the anchor to the driver;
inserting the free end of the driver, the loop, the two legs of the second suture, and the anchor into a hole in the bone; and
simultaneously applying tension on the loop and rotating the handle to screw the anchor into the hole and to secure the two legs of the second suture to the bone.

18. A system is provided for receiving a plurality of sutures and for securing at least one thereof in boney tissue using an anchor, the system comprising:
an elongated handle having first end and a second end generally opposite the first end and defining a central bore extending therethrough generally centered about a central axis, the central bore being configured to receive a first suture of the plurality of sutures;
an elongated driver connected to the handle and defining a central bore extending therethrough generally centered about the central axis and in communication with the central bore of the handle, the central bore of the driver being configured to receive the first suture;
at least one first cleat proximate the first end of the handle configured to receive and hold the first suture, and the handle being configured to rotate the driver upon rotation of the handle;
an elongated collar proximate the second end of the handle configured to receive the driver and defining a central bore extending therethrough generally centered about the central axis; and
at least one second cleat provided on the collar configured to receive and hold a second suture of the plurality of sutures.

19. The system of claim 18, further comprising the driver having a longitudinally-extending profile of a cross-section including at least one of a group consisting of star shape, a decagon shape, nonagon shape, an octagon shape, heptagon shape, hexagon shape, square shape, triangle shape, oval shape, symmetrical shape, asymmetrical shape, and polygonal shape

20. The system of claim 18, further comprising the collar being configured to move axially with respect to the driver.
